(19) United States
(12) Patent Application Publication
(10) Pub. No.: US 2003/0212319 A1
(43) Pub. Date: Nov. 13, 2003

(54) Health monitoring garment

(76) Inventor: Alan Remy Magill, London (GB)

Correspondence Address: Breiner & Breiner, L.L.C., P.O. Box 19290, Alexandria, VA, 22320-0290, US

(21) Appl. No.: 10/410,281

(22) Filed: Apr. 10, 2003

Related U.S. Application Data
(63) Continuation-in-part of application No. PCT/EP00/10011, filed on Oct. 10, 2000.

(51) Int. Cl. A61B 5/04

(52) U.S. Cl. 600/382; 600/388; 600/389

(57) ABSTRACT

A health monitoring garment which employs a means of conducting electricity from the surface of the skin, through the fibres of a fabric to another fabric which is removably attached to it and contains a microprocessor, telemetry and a power source to monitor and transmit EKG data of a person wearing the clothing, as illustrated in FIG. 2. Removability enables tile garment to be washed and the electronics to be kept separate from the washing and tumble drying process. The same system can be used in reverse to effect cardiac pacing or defibrillation or to deliver other forms of electronically conveyed healing such as tissue repair.

### FIELD OF INVENTION

The present invention relates to health monitoring garments, especially for electrophysiological cardiac and respiratory monitoring and for monitoring vital signs of a human or animal subject.

### BACKGROUND OF THE INVENTION

Electrophysiological cardiac monitoring of humans is usually referred to (in English) as the Electro Cardiogram (ECG), or the EKG in the USA. Electrophysiological respiratory monitoring is generally referred to as impedance monitoring. A third form of electrophysiological monitoring of the very small electrical impulses from human tissue, muscle or other body cells, which are usually measured by SQUID biomagnetometers to produce three-dimensional current distribution images, for example, of the heart, is known as Biomagnetic Computed Tomography.

There are many commonly used ambulatory methods of monitoring electrophysiological information such as the Holter, pendant, and credit card heart monitors. In addition, U.S. Pat. No. 3,954,100 discloses a flexible sensor pad, for collecting an EKG signal, which the subject sits or lies upon. It is also known to collect such signals using a number of electrodes held in contact with the skin of the subject by a chest-strap GB2291505, a belt U.S. Pat. No. 4,391,279, or a garment EP 0788811 A1 and U.S. Pat. No. 4,583,547. The garment disclosed in U.S. Pat. No. 4,583,547 also includes a conductive medium knitted or woven into the cloth which carries the signal to an external electrical apparatus. Additionally, some clothing includes microchips and fibre optics to ambulatorily monitor the heart, and WO 99/64657 discloses a garment produced using plastic optic fibres which can act as a “data bus” enabling other devices for gathering and/or processing signals from sensors to be connected to the garment. However, all these current systems of electrophysiological monitoring are not wholly non-invasive in that they need to be regarded as special medical devices. The wearing of a medical device has undesirable psychological implications attached to it in that few people like to admit to either themselves or others that they require a machine as part of their everyday lives. Additionally, there are the ergonomic restrictions of the discomfort and inconvenience associated with the attachment of electrodes, gels, straps and belts to the human body. Hence, the traditional ECG or respiratory monitor creates an undesirable intrusion into everyday life.

### OBJECTS AND BRIEF DESCRIPTION OF THE INVENTION

It is one of the objects of the present invention to monitor electrophysiology in a less obtrusive, cumbersome and inconvenient manner than with known devices.

According to the present invention, there is provided a device for monitoring electrophysiological vital signs of a subject comprising a garment with a plurality of electrodes on the inside of the garment, and a processing means connected to the electrodes arid which can monitor these vital signs. The electrodes are comprised of internal loops of conductive yarn which, when the garment is in use, contact the subject's skin such that electrical impulses present on the skin of the subject are passed via the electrodes to the processing means. The garment may be in the form of a vest or undershirt for wear by the subject and which has stretch both longitudinally and laterally of the subject's body to fit the body closely. It is constructed from conductive and non-conductive yarns. These can be knitted by such machines as a Double Loop Automatic Needle Selection Circular Knitting Machine. These conductive yarns may be knitted in the form of loops on the outside of the garment immediately opposite and integrally conjoined with corresponding internal conductive loops on the inside of the garment where the internal conductive loops, touch the human or animal skin when the garment is worn. The internal and external loops are entwined during the knitting process so as to transfer electrical impulses from the inside to the outside of the garment. This provides an alternative to the use of adhesive electrodes used by traditional ECG monitors. The knitted conductive yarns are hereinafter referred to as “knitted electrodes”. In order to insulate one pair of knitted electrodes from another, a separate group of conductive yarns is fed into the circular knitting machine for each pair of electrodes, interspaced with non-conductive yarns.

The processing means may be incorporated in a sensor pad in the form of a common carrier, hereinafter referred to as an “ECG patch”, which is removably attachable to the outside of the garment, in such a way that the processing means may be connected to the electrodes. The ECG patch may have attachment means on its underside in the form of part of a burr fastener, e.g. as sold under the Registered Trade Mark “Velcro”. The hooks of the burrs fastener are preferably located on the ECG patch and can be made by the same knitting or moulding process as that used to make standard burr fastener fastening means. However, it is preferred in the case of an ECG patch to make the hooks from a conductive material such as an electrically conductive polymer or polymers loaded with conductive particulates (when the hooks are made by moulding) or from conductive yarns made from such materials as stainless steel, copper, polymer and carbon fibre (where the hooks are made from knitted loops cut asymmetrically). Whilst burr fastener attachment means are a preferred embodiment, alternative removable attachment means may be used, for example, poppers, buttons, string or a randomly constructed mass of entwined conductive yarn which can engage around loops of the garment textile structure. Regardless of the particular type of removable attachment means utilised, it is important that at least part of the material used is electroconductive so, for example, conductive yarn or an electroconductive substrate made from cloth, paper or rubber may be used.

Contact between the knitted electrodes and e.g. conductive hooks located on the underside of the ECG patch enables the electrical impulses present on the human skin to be passed to a microprocessor and wire-free transmission means located inside the ECG patch.

The benefit of using the common carrier hook and loop wire free transmission means, the ECG patch, is that it can be used as a releasable, tear-off patch to be removed prior to washing. This enables the garment to be washed, tumble-dried and ironed without exposing any electronic package to undue water ingress, heat or friction, thereby allowing the garment to be treated like any other normal, washable, everyday garment.

The ECG patch may incorporate sufficient electronic processing power and the appropriate algorithms to give a warning of impending severe illness such as a heart attack or asthma attack; the warning can be in the form of a flashing light and/or audible warnings emanating from the ECG patch.

A transformer may be incorporated in the ECG patch, together with an auxiliary electric power source which, upon receiving a command from the microprocessor, can deliver electrical shocks to the subject via the knitted electrodes to which it is attached. These electric shocks may be mild so as to provide a cardiac pacing function, or they may be, more powerful, such as 200-300 volts, to provide a defibrillating function. (Recent research has demonstrated that low power shocks can be used to restore normal heart rhythm in a fibrillating or arrhythmetic patient instead of high power 2000 to 30000 volt shocks). Additionally, battery power could be provided by an auxiliary battery located in the ECG patch, or by wire connection to an auxiliary battery located somewhere on the subject, such as in a pocket.

The ECG patch may include a wire-free transmission and receiving means such as radio or infrared system in order to send and receive cardio-respiratory data to a palmtop computer/mobile telephone worn by the subject. By this means data can be processed using the greater processing power available in a palmtop computer than that available in the ECG patch. Additionally, the mobile telephone can be used to send data for remote analysis by ail appropriate science or medical research centre or for review by a physician. Such a centre could access and cross-reference data from hundreds or thousands of patients, downloading millions of hours of cardiac events and thereby provide the epidemiological data for long-term research and development necessary for the compilation of universally applicable warning signals for critical conditions such as sudden cardiac death or asthma attacks. The mobile telephone can be designed to receive data from the physician or research centre which enables the palmtop computer to update or modify pacing or defibrillating instructions effected by the ECG patch.

If desired the palmtop computer can be fitted with a removable memory card which can be used to post cardiac and respiratory data to a physician in the event that the telephone system does not function correctly.

It is also conceivable that separate defibrillating knitted electrodes may be incorporated in the ECG patch whose function is only to administer electric shocks to the patient. Alternatively, in the event that a patient is not suitable for low voltage defibrillation, a standard high voltage adhesive defibrillation patch can be adhered to the patient and attached by wire to control electronics in the ECG patch and by other wires to an appropriate high voltage power source

The conductivity of the contact between the knitted electrodes and the skin may be increased, if desired, above the normal level inherent in conductive yarns such as for example polymer yarns, conductive stainless steel yarns, organic conductive yarns (i.e. made from organic material that is grown and harvested), or conductive polymer yarns entwined with cotton yarns which, when placed against the skin in the manner described by “Knitted Electrodes”, absorb sweat and provide adequate ECG and respiratory signals (as proved by experiment by the applicant). This improvement in conductivity may be achieved by a fabric-finishing process such as cutting and brushing the inside loops of the knitted electrodes and/or the application of an ideally water-based spray-on conductive gel of a medium similar to anti-perspirant. This adheres to the fibres, is deformed by the pressure of the garment against the skin and increases the area of conductivity between each fibre and the skin.

The ECG patch may also include a remotely programmable electronic personal information tag to record the user's name, address and primary health data.

The invention is particularly useful for the long-term, continuous and 24 a day hour monitoring of heart: patients, particularly those who may be susceptible to sudden cardiac death of which some 800,000 people die every year in Europe and the USA. By using the invention, the subjects may be monitored by a vest or undershirt that to them appears very similar to normal apparel, with none of the discomfort, stigma or psychological burdens associated with traditional ECG or respiriatory monitors. By wearing the invention—the ECG vest continuously, particularly at night when most of the potentially fatal cardiac events are known to occur, the computer databases linked to it, either remotely by telephone or quasi remotely by the palmtop computer worn by the patient or directly by an onboard ASIC in the ECG patch (the ASIC being equipped with the appropriate microprocessor and event-identifying algorithms), the invention has a variety of life-preserving opportunities to process sufficient data to forewarn patients of an imminent heart attack and, if necessary, deliver arrhythmia-regulating or defibrillating electric current.

### DETAILED DESCRIPTION OF PRESENTLY PREFERRED EMBODIMENTS OF THE INVENTION

Methods and systems for monitoring electrophysiological and electro respiratory vital signs of a subject using a device in accordance with the present invention will now be described, by way of example, with reference to the accompanying drawings in which:

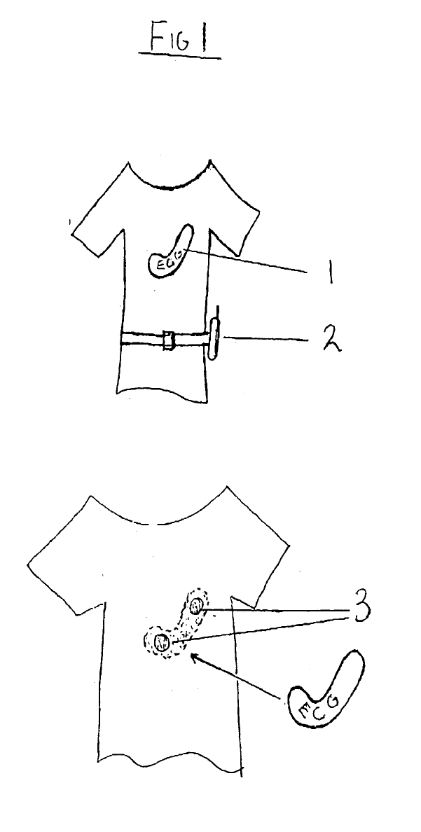

FIG. 1 is the general embodiment of an ECG vest where 1 is the releasable common carrier, the ECG patch, 2 is the patient's mobile phone and computer with which it communicates and 3 denotes the knitted electrodes beneath an ECG patch that has been removed. For the purpose of simplicity, only two of several areas of conductive loops are shown. These conductive loop areas could encompass the body if required.

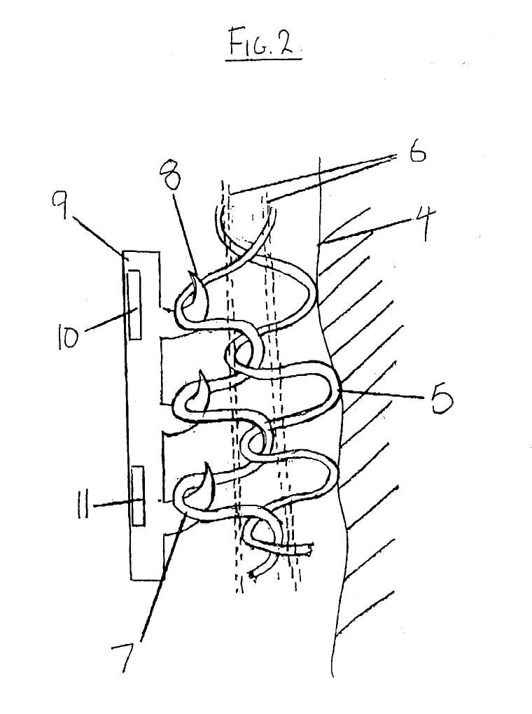

FIG. 2 is a cross-sectional representation to illustrate how the electrical impulses present on the surface of human skin 4, can be passed into a knitted or woven garment configuration comprising internal conductive loops 5 which touch the skin, foundation loops 6 made from a non-conductive yarn which hold the internal and external loops together, and external conductive loops 7. These are attached to conductive hooks located on a releasable patch 9 which contains electronic parts including an ASIC 10 and a battery 11.

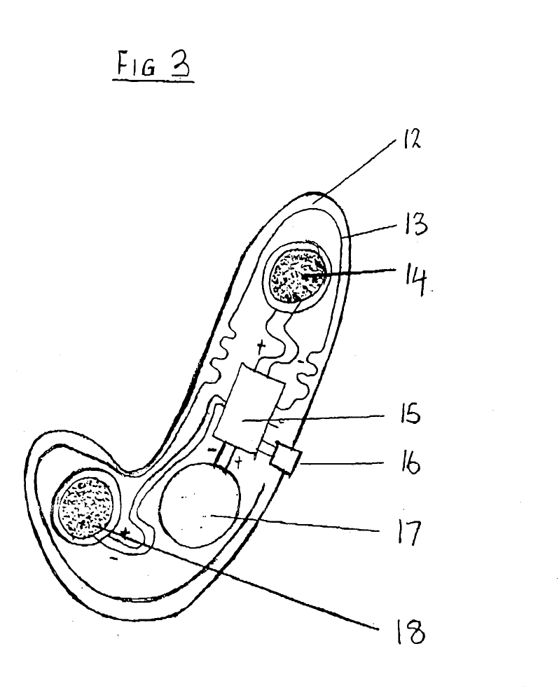

FIG. 3 is a plan view of the releasable ECG patch wherein 12 is the periphery of the patch which can be of any shape or design suitable to encompass the knitted electrodes. An antenna 13 is connected to an ASIC 15, an application specific integrated circuit and transformer containing electronic elements sufficient to transmit the ECG and respiratory impedance signals, to receive commands from a nearby mobile phone or to receive internal commands generated by ASIC onboard algorithms and to act on those commands in respect to the provision of electric shocks via two conductive hooked pads 14 and 18. An electric socket 16 is provided with sufficient connections to enable the ASIC 15 to receive auxiliary electrical power and to communicate defibrillating instructions to standard adhesive defibrillator electrodes which may be applied to the patient in the event that the low voltage defibrillation capacity of the invention needs to be supplemented by high voltage defibrillation. The battery power supply to the ASIC is denoted 17. Wiring connections are shown by lines with + and − symbols attached to them.

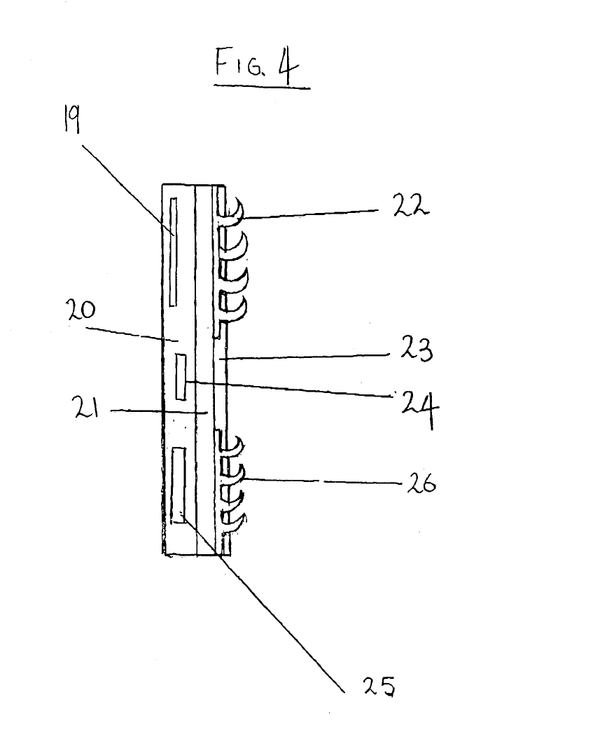

FIG. 4 is a cross-sectional representation of the ECG patch wherein 19 is a programmable electronic label to hold the patient's personal data; 20 is an outside protective layer which may have designs and decorations on it, 21 is a first inner electrical insulation layer, 22 is one of the conductive hooked pads, 23 is a second non-conductive layer which may also be constructed as part of a hook and loop fastening means, but of the non-conductive type. An ASIC is denoted 24, a battery 25 and a second conductive hooked pad 26. The pads and electronic parts may be stuck with non-conductive adhesive attached to the outside protective layer. Materials shown at 20, 21 and 23 can be made from elastic components so that they stretch and the electrical connections between each conductive pad 22, 26 and the ASIC 24 can be designed so that they too can accept the flexing movement of this ECG and respiratory patch to accommodate the chest expansion and contraction as the wearer breathes. Alternatively, the ECG patch may be made from materials shown at 20, 21 and 23 which have a latticework of holes so enabling the patch to stretch to conform with the movement of the human or animal subject.

1. A device for monitoring electrophysiological vital signs of a subject comprising a garment with a plurality of electrodes, on the inside of the garment, and a processing means which can monitor these vital signs, wherein the electrodes are comprised of internal loops of conductive yarn which, when the garment is in use, contact the subject's skin such that electrical impulses present on the skin of the subject are passed via the electrodes to thc processing means.

2. The device of claim 1, wherein the garment further comprises external loops of conductive yarn which are entwined with the internal loops whereby electrical impulses may be transferred from the inside to the outside of the garment.

3. The device of claim 1 or 2, wherein the conductive yarn is made from stainless steel, copper, polymer, carbon fibre or organic material.

4. The device of claim 1 or 2, wherein each electrode is insulated from other electrodes by being interspaced with non-conductive yarn.

5. The device of claim 1, wherein the processing means are incorporated on a sensor pad which is removably attachable to the garment by an electronically conductive attachment means.

6. The device of claim 5, wherein the electronically conductive attachment means is a burr fastener.

7. The device of claim 6, wherein the hooks of the burr fastener are fabricated from an electrically conductive polymer, or a polymer loaded with conductive particulates.

8. The device of claim 1, wherein the processing means is connected to a wire free transmission and receiving means such that it can send cardio-respiratory date for remote analysis and receive warning data.

9. The device of claim 8, wherein the wire-free transmission and receiving means is selected from a radio or infrared system or a mobile phone.

10. The device of claim 1, and including means adapted to emit an audible or visual warning signal, and wherein the processing means is adapted to analyse incoming data using appropriate event-identifying algorithms so that impending severe illness will cause the device to emit a warning in the form of such a signal.

11. The device of claim 1, and including, connected to the processing means, a transformer and an auxiliary electrical power source, the transformer being attached to the electrodes such that on receiving a command from the processing means the transformer can deliver an electric shock to the subject via the electrodes.

12. The device of claim 1, and including, connected to the processing means, a transformer, an auxiliary electrical power source, and a plurality of separate defibrillating electrodes attached to the transformer, whereby on receiving a command from the processing means the transformer can deliver an electric shock to the subject via the separate defibrillating electrodes.

13. The device of claim 1, wherein the electrodes are brushed to improve their electrical contact with the skin.

14. The device of claim 1, wherein the electrodes are cut to improve their electrical contact with the skin.

15. The device of claim 1, wherein the electrodes have a conductive gel applied to them to improve electrical contact with the skin.
